(19) United States
(12) Patent Application Publication
(10) Pub. No.: US 2011/0306967 A1
(43) Pub. Date: Dec. 15, 2011

(54) COOLING CONFIGURATIONS FOR ELECTROSURGICAL INSTRUMENTS

(76) Inventors: Gwendolyn P. Payne, Cincinnati, OH (US); Scott A. Woodruff, Cincinnati, OH (US); Donna L. Korvick, Maineville, OH (US); David K. Norvell, Monroe, OH (US)

(21) Appl. No.: 12/797,861

(22) Filed: Jun. 10, 2010

(51) Int. Cl. A61B 18/14

(52) U.S. Cl. 606/41

(57) ABSTRACT

In various embodiments, a surgical instrument is provided that may comprise an end effector comprising at least one energy delivery surface and cooling means for cooling at least a portion of the end effector. For example, in at least one embodiment, a surgical instrument may comprise a handle, an elongate shaft operably coupling the handle to the end effector, and a pump operably coupled to the handle. In such embodiments, the pump may be configured to cause a fluid to move through the elongate shaft and over at least a portion of the end effector. Additionally, in at least one embodiment, a surgical kit is provided that may comprise a surgical instrument and a cap configured to receive at least a portion of the surgical instrument's end effector. In such embodiments, the cap may be sized and configured to receive at least a portion of the end effector. Moreover, the cap may be sized and configured to fit through a trocar.

### BACKGROUND

The present disclosure is directed to medical devices and methods, and, more particularly, to electrosurgical instruments and methods for sealing and transecting tissue.

In various circumstances, a surgical instrument can be configured to apply energy to tissue in order to treat and/or destroy the tissue. In certain circumstances, a surgical instrument can comprise one or more electrodes which can be positioned against and/or positioned relative to the tissue such that electrical current can flow from one electrode, through the tissue, and to the other electrode. The surgical instrument can comprise an electrical input, a supply conductor electrically coupled with the electrodes, and/or a return conductor which can be configured to allow current to flow from the electrical input, through the supply conductor, through the electrodes and the tissue, and then through the return conductor to an electrical output, for example. In various circumstances, heat can be generated by the current flowing through the tissue, wherein the heat can cause one or more hemostatic seals to form within the tissue and/or between tissues. Such embodiments may be particularly useful for sealing blood vessels, for example. The surgical instrument can also comprise a cutting member that can be moved relative to the tissue and the electrodes in order to transect the tissue.

By way of example, energy applied by a surgical instrument may be in the form of radio frequency (“RF”) energy. RF energy is a form of electrical energy that may be in the frequency range of 300 kilohertz (kHz) to 1 megahertz (MHz). In application, RF surgical instruments transmit low frequency radio waves through electrodes, which cause ionic agitation, or friction, increasing the temperature of the tissue. Since a sharp boundary is created between the affected tissue and that surrounding it, surgeons can operate with a high level of precision and control, without much sacrifice to the adjacent normal tissue. The low operating temperatures of RF energy enables surgeons to remove, shrink or sculpt soft tissue while simultaneously sealing blood vessels. RF energy works particularly well on connective tissue, which is primarily comprised of collagen and shrinks when contacted by heat.

Further, in various open and laparoscopic surgeries, it may be necessary to coagulate, seal or fuse tissues. One means of sealing tissue relies upon the application of electrical energy to tissue captured within an end effector of a surgical instrument in order to cause thermal effects within the tissue. Various mono-polar and bi-polar RF jaw structures have been developed for such purposes. In general, the delivery of RF energy to the captured tissue elevates the temperature of the tissue and, as a result, the energy can at least partially denature proteins within the tissue. Such proteins, such as collagen, for example, may be denatured into a proteinaceous amalgam that intermixes and fuses, or “welds,” together as the proteins renature. As the treated region heals over time, this biological “weld” may be reabsorbed by the body's wound healing process.

In certain arrangements of a bi-polar radiofrequency (RF) jaw, the surgical instrument can comprise opposing first and second jaws, wherein the face of each jaw can comprise an electrode. In use, the tissue can be captured between the jaw faces such that electrical current can flow between the electrodes in the opposing jaws and through the tissue positioned therebetween. Such instruments may have to seal or “weld” many types of tissues, such as anatomic structures having walls with irregular or thick fibrous content, bundles of disparate anatomic structures, substantially thick anatomic structures, and/or tissues with thick fascia layers such as large diameter blood vessels, for example. With particular regard to sealing large diameter blood vessels, for example, such applications may require a high strength tissue weld immediately post-treatment.

The foregoing discussion is intended only to illustrate the present field and should not be taken as a disavowal of claim scope.

### SUMMARY

In various embodiments, a surgical kit is provided. In at least one embodiment, the surgical kit can comprise a surgical instrument comprising an end effector and a cap comprising a body including a first end and a second end. In these embodiments, the body can define a cavity and the first end can define an opening to the cavity. Additionally, in these embodiments, the cavity can be sized and configured to receive at least a portion of the end effector. Moreover, the body can be sized and configured to fit through a trocar.

In various embodiments a surgical instrument is provided. In at least one embodiment, the surgical instrument can comprise a handle, an end effector and an elongate shaft operably coupling the handle to the end effector, and a pump operably coupled to the handle. In these embodiments, the pump can be configured to cause a fluid to move through the elongate shaft and over at least a portion of the end effector.

In at least one embodiment, a surgical instrument is provided that can comprise an end effector comprising at least one energy delivery surface, and cooling means for cooling at least a portion of the end effector.

The foregoing discussion should not be taken as a disavowal of claim scope.

### FIGURES

Various features of the embodiments described herein are set forth with particularity in the appended claims. The various embodiments, however, both as to organization and methods of operation, together with advantages thereof, may be understood in accordance with the following description taken in conjunction with the accompanying drawings as follows.

FIG. 1 is a perspective view of a surgical kit including a surgical instrument and a cap according to a non-limiting embodiment.

FIG. 2 is a side view of a handle of the surgical instrument of FIG. 1 with a half of a handle body removed to illustrate some of the components therein.

FIG. 3 is a perspective view of an end effector of the surgical instrument of FIG. 1 illustrated in an open configuration; the distal end of a cutting member is illustrated in a retracted position.

FIG. 4 is a perspective view of the end effector of the surgical instrument of FIG. 1 illustrated in a closed configuration; the distal end of the cutting member is illustrated in a partially advanced position.

FIG. 5 is a perspective sectional view of a portion of a cutting member of the surgical instrument of FIG. 1; the cutting member is shown at least partially shaped like an I-beam.

FIG. 6 is a perspective view of the end effector of the surgical instrument and cap of FIG. 1 with the cap placed over the surgical instrument's end effector.

FIG. 7 is a side view of a handle of a surgical instrument with a half of a handle body removed to illustrate some of the components therein according to a non-limiting embodiment; a pump of the surgical instrument is shown drawing air into and through the instrument.

FIG. 8 is a perspective view of an end effector of the surgical instrument of FIG. 7 illustrated in an open configuration with air being blown over a portion of the end effector.

FIG. 9 is a side view of a handle of a surgical instrument with a half of a handle body removed to illustrate some of the components therein according to a non-limiting embodiment; a pump of the surgical instrument is shown drawing air through and out of the instrument.

FIG. 10 is a perspective view of an end effector of the surgical instrument of FIG. 9 illustrated in an open configuration with air being vacuumed over a portion of the end effector.

FIG. 11 is a side view of a handle of a surgical instrument with a half of a handle body removed to illustrate some of the components therein according to a non-limiting embodiment; a pump of the surgical instrument is shown moving liquid from a reservoir through the instrument.

FIG. 12 is a perspective view of an end effector of the surgical instrument of FIG. 11 illustrated in an open configuration with liquid being sprayed over a portion of the end effector.

FIG. 13 is a perspective view of an end effector of the surgical instrument of FIG. 11 illustrated in a closed configuration with liquid being sprayed over a portion of the end effector.

FIG. 14 is a side view of a jaw of an end effector of a surgical instrument according to a non-limiting embodiment; a heat sink is shown located within the jaw.

FIG. 15 is a partial perspective view of an end effector of a surgical instrument according to a non-limiting embodiment; a heat sink is shown located within the end effector.

FIG. 16 is a side view of a handle of a surgical instrument with a half of a handle body removed to illustrate some of the components therein according to a non-limiting embodiment.

FIG. 17 is a perspective view of an end effector of the surgical instrument of FIG. 16 illustrated in a closed configuration with a thermocouple located within the end effector.

Corresponding reference characters indicate corresponding parts throughout the several views. The exemplifications set out herein illustrate various embodiments, in one or more forms, and such exemplifications are not to be construed as limiting the scope of the claims in any manner.

### DETAILED DESCRIPTION

Various embodiments are directed to apparatuses, systems, and methods for the treatment of tissue. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

The entire disclosures of the following non-provisional United States patents are hereby incorporated by reference herein:

U.S. Pat. No. 7,381,209, entitled ELECTROSURGICAL INSTRUMENT;

U.S. Pat. No. 7,354,440, entitled ELECTROSURGICAL INSTRUMENT AND METHOD OF USE;

U.S. Pat. No. 7,311,709, entitled ELECTROSURGICAL INSTRUMENT AND METHOD OF USE;

U.S. Pat. No. 7,309,849, entitled POLYMER COMPOSITIONS EXHIBITING A PTC PROPERTY AND METHODS OF FABRICATION;

U.S. Pat. No. 7,220,951, entitled SURGICAL SEALING SURFACES AND METHODS OF USE;

U.S. Pat. No. 7,189,233, entitled ELECTROSURGICAL INSTRUMENT; U.S. Pat. No. 7,186,253, entitled ELECTROSURGICAL JAW STRUCTURE FOR CONTROLLED ENERGY DELIVERY;

U.S. Pat. No. 7,169,146, entitled ELECTROSURGICAL PROBE AND METHOD OF USE;

U.S. Pat. No. 7,125,409, entitled ELECTROSURGICAL WORKING END FOR CONTROLLED ENERGY DELIVERY; and

U.S. Pat. No. 7,112,201, entitled ELECTROSURGICAL INSTRUMENT AND METHOD OF USE.

The following co-pending United States patent applications, filed on even date herewith, are also hereby incorporated by reference herein:

U.S. patent application Ser. No. ______ (Attorney Docket No. END6655USNP/090293), entitled ELECTROSURGICAL INSTRUMENT COMPRISING SEQUENTIALLY ACTIVATED ELECTRODES;

U.S. patent application Ser. No. ______ (Attorney Docket No. END6656USNP/090294), entitled ELECTROSURGICAL INSTRUMENT EMPLOYING A THERMAL MANAGEMENT SYSTEM; and

U.S. patent application Ser. No. ______ (Attorney Docket No. END6658USNP/090296), entitled HEAT MANAGEMENT CONFIGURATIONS FOR CONTROLLING HEAT DISSIPATION FROM ELECTROSURGICAL INSTRUMENTS.

Various embodiments of systems and methods relate to creating thermal “welds” or “fusion” within native tissue volumes. The alternative terms of tissue “welding” and tissue “fusion” may be used interchangeably herein to describe thermal treatments of a targeted tissue volume that result in a substantially uniform fused-together tissue mass, for example, in welding blood vessels that exhibit substantial burst strength immediately post-treatment. The strength of such welds is particularly useful for (i) permanently sealing blood vessels in vessel transection procedures; (ii) welding organ margins in resection procedures; (iii) welding other anatomic ducts wherein permanent closure is required; and also (iv) for performing vessel anastomosis, vessel closure or other procedures that join together anatomic structures or portions thereof. The welding or fusion of tissue as disclosed herein is to be distinguished from “coagulation”, “hemostasis” and other similar descriptive terms that generally relate to the collapse and occlusion of blood flow within small blood vessels or vascularized tissue. For example, any surface application of thermal energy can cause coagulation or hemostasis—but does not fall into the category of “welding” as the term is used herein. Such surface coagulation does not create a weld that provides any substantial strength in the treated tissue.

At the molecular level, the phenomena of truly “welding” tissue as disclosed herein may result from the thermally-induced denaturation of collagen and other protein molecules in a targeted tissue volume to create a transient liquid or gel-like proteinaceous amalgam. A selected energy density is provided in the targeted tissue to cause hydrothermal breakdown of intra- and intermolecular hydrogen crosslinks in collagen and other proteins. The denatured amalgam is maintained at a selected level of hydration—without desiccation—for a selected time interval which can be very brief. The targeted tissue volume is maintained under a selected very high level of mechanical compression to insure that the unwound strands of the denatured proteins are in close proximity to allow their intertwining and entanglement. Upon thermal relaxation, the intermixed amalgam results in protein entanglement as re-crosslinking or renaturation occurs to thereby cause a uniform fused-together mass.

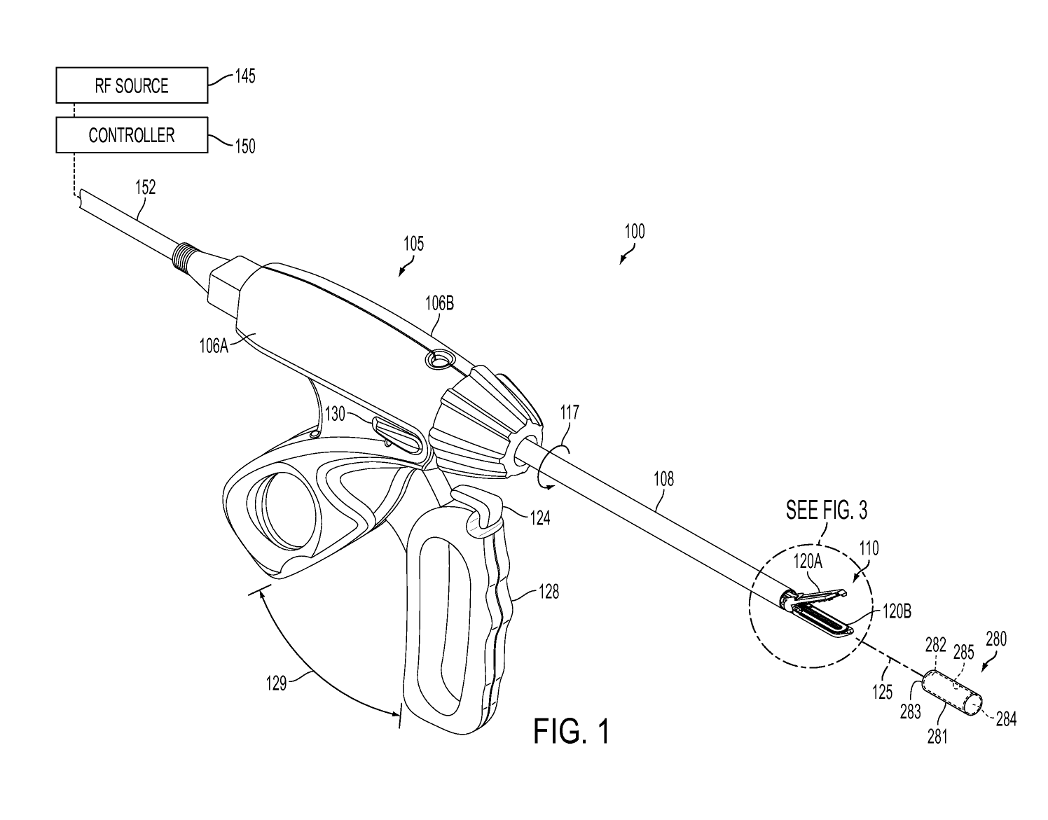

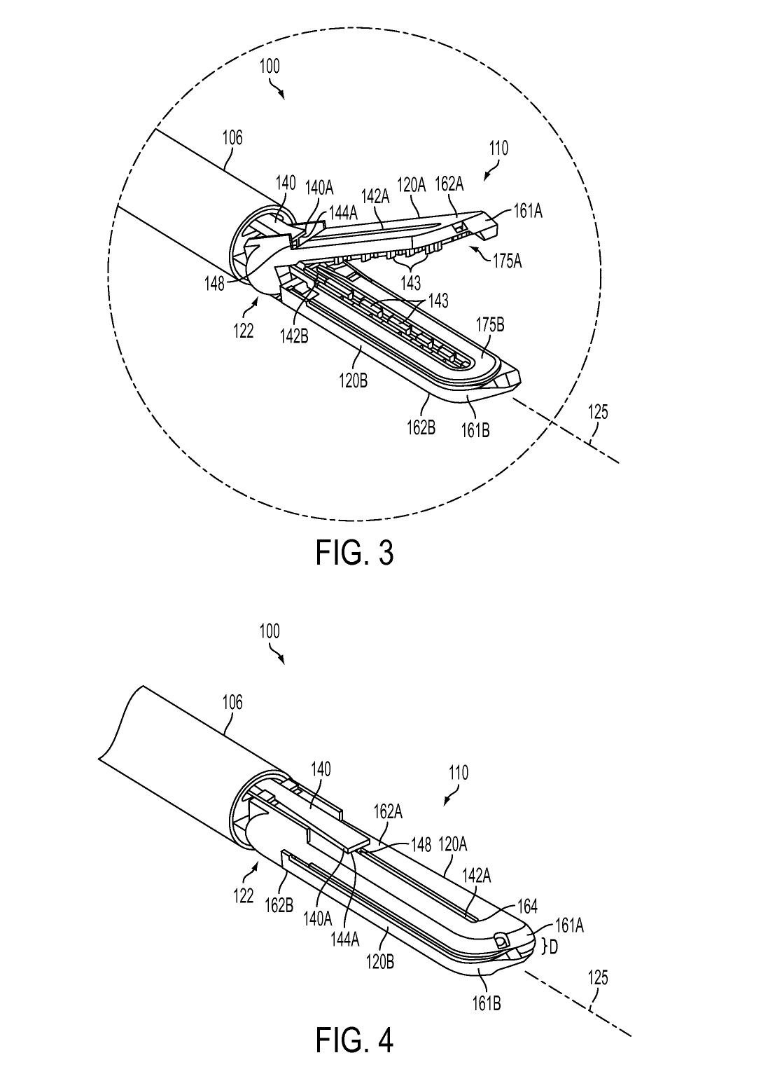

A surgical instrument can be configured to supply energy, such as electrical energy, ultrasonic energy, and/or heat energy, for example, to the tissue of a patient. For example, various embodiments disclosed herein provide electrosurgical jaw structures adapted for transecting captured tissue between the jaws and for contemporaneously welding the captured tissue margins with controlled application of RF energy. In more detail, in various embodiments, referring now to FIG. 1, an electrosurgical instrument 100 is shown. Surgical or electrosurgical instrument 100 can comprise a proximal handle 105, a distal working end or end effector 110 and an introducer or elongate shaft 108 disposed in-between. End effector 110 may comprise a set of openable-closeable jaws with straight or curved jaws—an upper first jaw 120A and a lower second jaw 120B. First jaw 120A and second jaw 120B may each comprise an elongate slot or channel 142A and 142B (see FIG. 3), respectively, disposed outwardly along their respective middle portions. First jaw 120A and second jaw 120B may be coupled to an electrical source 145 and a controller 150 through electrical leads in cable 152. Controller 150 may be used to activate electrical source 145. In various embodiments, the electrical source 145 may comprise an RF source, an ultrasonic source, a direct current source, and/or any other suitable type of electrical energy source, for example.

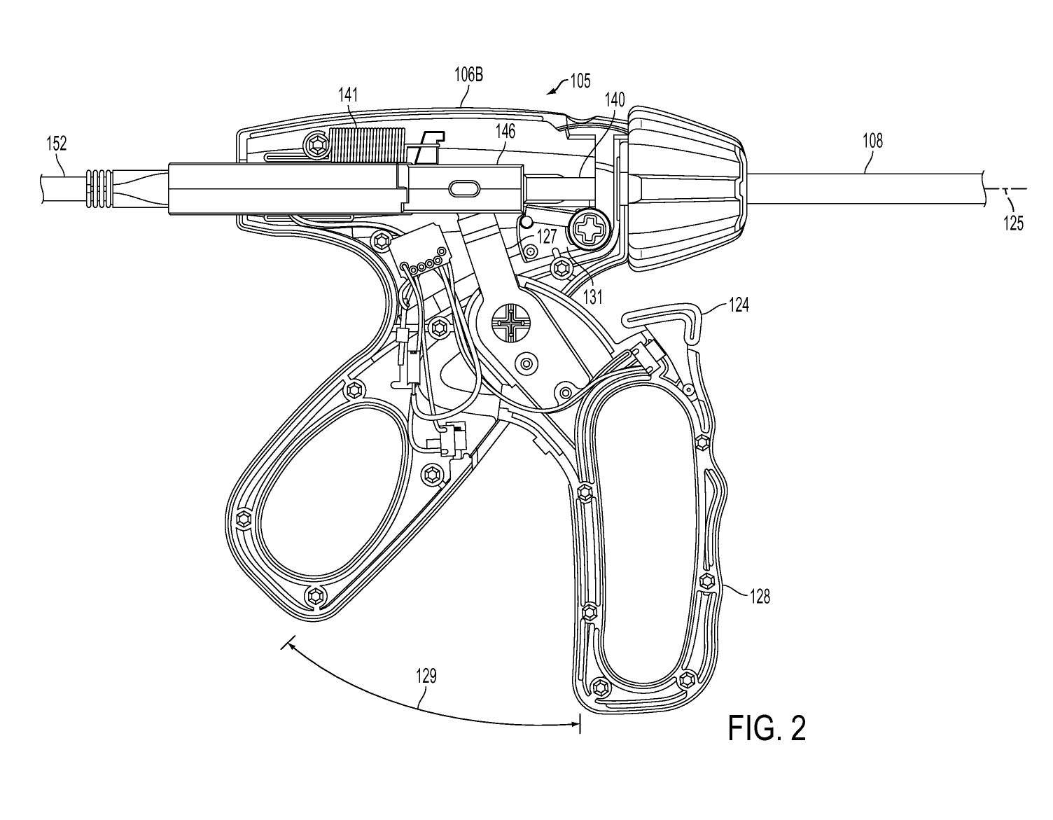

Moving now to FIG. 2, a side view of the handle 105 is shown with half of a first handle body 106A (see FIG. 1) removed to illustrate some of the components within second handle body 106B. Handle 105 may comprise a lever arm 128 which may be pulled along a path 129. Lever arm 128 may be coupled to a movable cutting member 140 disposed within elongate shaft 108 by a shuttle 146 operably engaged to an extension 127 of lever arm 128. The shuttle 146 may further be connected to a biasing device, such as spring 141, which may also be connected to the second handle body 106B, to bias the shuttle 146 and thus the cutting member 140 in a proximal direction, thereby urging the jaws 120A and 120B to an open position as seen in FIG. 1. Also, referring to FIGS. 1 and 2, a locking member 131 (see FIG. 2) may be moved by a locking switch 130 (see FIG. 1) between a locked position, where the shuttle 146 is substantially prevented from moving distally as illustrated, and an unlocked position, where the shuttle 146 may be allowed to freely move in the distal direction, toward the elongate shaft 108. The handle 105 can be any type of pistol-grip or other type of handle known in the art that is configured to carry actuator levers, triggers or sliders for actuating the first jaw 120A and second jaw 120B. Elongate shaft 108 may have a cylindrical or rectangular cross-section and can comprise a thin-wall tubular sleeve that extends from handle 105. Elongate shaft 108 may include a bore extending therethrough for carrying actuator mechanisms, for example, cutting member 140, for actuating the jaws and for carrying electrical leads for delivery of electrical energy to electrosurgical components of end effector 110.

End effector 110 may be adapted for capturing, welding and transecting tissue. First jaw 120A and second jaw 120B may close to thereby capture or engage tissue about a longitudinal axis 125 defined by cutting member 140. First jaw 120A and second jaw 120B may also apply compression to the tissue. Elongate shaft 108, along with first jaw 120A and second jaw 120B, can be rotated a full 360° degrees, as shown by arrow 117, relative to handle 105 through, for example, a rotary triple contact. First jaw 120A and second jaw 120B can remain openable and/or closeable while rotated.

FIGS. 3 and 4 illustrate perspective views of end effector 110. FIG. 3 shows end effector 110 in an open configuration and FIG. 4 shows end effector 110 in a closed configuration. As noted above, the end effector 110 may comprise the upper first jaw 120A and the lower second jaw 120B. Further, the first jaw 120A and second jaw 120B may each have tissue-gripping elements, such as teeth 143, disposed on the inner portions of first jaw 120A and second jaw 120B. First jaw 120A may comprise an upper first jaw body 161A with an upper first outward-facing surface 162A and an upper first energy delivery surface 175A. Second jaw 120B may comprise a lower second jaw body 161B with a lower second outward-facing surface 162B and a lower second energy delivery surface 175B. First energy delivery surface 175A and second energy delivery surface 175B may both extend in a “U” shape about the distal end of end effector 110.

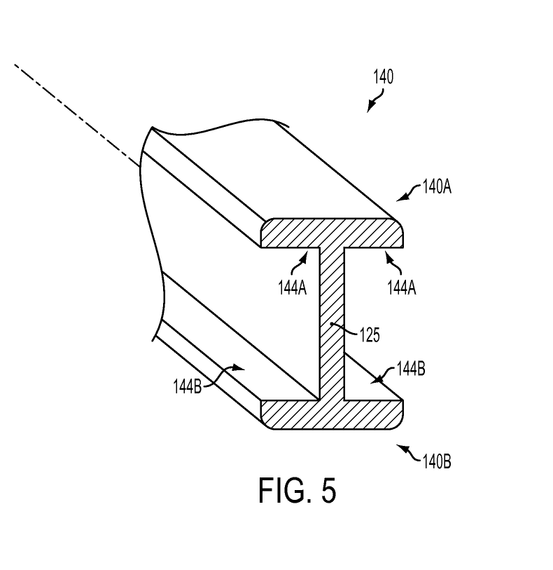

Referring briefly now to FIG. 5, a portion of cutting member 140 is shown. The lever arm 128 of handle 105, see FIG. 2, may be adapted to actuate cutting member 140 which also functions as a jaw-closing mechanism. For example, cutting member 140 may be urged distally as lever arm 128 is pulled proximally along path 129 via shuttle 146, seen in FIG. 2 and discussed above. The cutting member 140 may comprise one or several pieces, but in any event, may be movable or translatable with respect to the elongate shaft 108 and/or jaws 120A, 120B. Also, in at least one embodiment, the cutting member 140 may be made of 17-4 precipitation hardened stainless steel. The distal end of cutting member 140 may comprise a flanged “I”-beam configured to slide within channels 142A and 142B in jaws 120A and 120B. Cutting member 140 may slide within channels 142A, 142B to open and close first jaw 120A and second jaw 120B. The distal end of cutting member 140 may also comprise upper flange or “c”-shaped portion 140A and lower flange or “c”-shaped portion 140B. The flanges 140A and 140B respectively define inner cam surfaces 144A and 144B for engaging outward facing surfaces of first jaw 120A and second jaw 120B. The opening-closing of jaws 120A and 120B can apply very high compressive forces on tissue using cam mechanisms which may include reciprocating “I-beam” cutting member 140 and the outward facing surfaces 162A, 162B of jaws 120A, 120B.

More specifically, referring now to FIGS. 3-5, collectively, inner cam surfaces 144A and 144B of the distal end of cutting member 140 may be adapted to slidably engage first outward-facing surface 162A and second outward-facing surface 162B of first jaw 120A and second jaw 120B, respectively. Channel 142A within first jaw 120A and channel 142B within second jaw 120B may be sized and configured to accommodate the movement of cutting member 140, which may comprise a tissue-cutting element, for example, a sharp distal edge. FIG. 4, for example, shows the distal end of cutting member 140 advanced at least partially through channels 142A and 142B (see FIG. 3). The advancement of cutting member 140 can close end effector 110 from the open configuration shown in FIG. 3. In the closed position shown by FIG. 4, upper first jaw 120A and lower second jaw 120B define a gap or dimension D between the first energy delivery surface 175A and second energy delivery surface 175B of first jaw 120A and second jaw 120B, respectively. Dimension D equals from about 0.0005″ to about 0.005″ and preferably between about 0.001″ to about 0.002″. Also, the edges of first energy delivery surface 175A and second energy delivery surface 175B may be rounded to prevent the dissection of tissue.

Referring now to FIGS. 1 and 3, end effector 110 may be coupled to electrical source 145 and controller 150. First energy delivery surface 175A and second energy delivery surface 175B may likewise each be coupled to electrical source 145 and controller 150. First energy delivery surface 175A and second energy delivery surface 175B may be configured to contact tissue and delivery electrosurgical energy to engaged tissue which is adapted to seal or weld the tissue. Controller 150 can regulate the electrical energy delivered by electrical source 145 which in turn delivers electrosurgical energy to first energy-delivery surface 175A and second energy-delivery surface 175B. The energy delivery may be initiated by an activation button 124 operably engaged with lever arm 128 and in electrical communication with controller 150 via cable 152. As mentioned above, the electrosurgical energy delivered by electrical source 145 may comprise radiofrequency (RF) energy. Further, the opposing first and second energy delivery surfaces 175A and 175B may carry variable resistive positive temperature coefficient (PTC) bodies that are coupled to electrical source 145 and controller 150. Additional details regarding electrosurgical end effectors, jaw closing mechanisms, and electrosurgical energy-delivery surfaces are described in the following U.S. patents and published patent applications, all of which are incorporated herein in their entirety by reference and made a part of this specification: U.S. Pat. Nos. 7,381,209; 7,311,709; 7,220,951; 7,189,233; 7,186,253; 7,125,409; 7,112,201; 7,087,054; 7,083,619; 7,070,597; 7,041,102; 7,011,657; 6,929,644; 6,926,716; 6,913,579; 6,905,497; 6,802,843; 6,770,072; 6,656,177; 6,533,784; and 6,500,176; and U.S. Pat. App. Pub. Nos. 2010/0036370 and 2009/0076506.

In various embodiments, it may be desirable to cool an end effector such that when energy is delivered to the end effector, as described above with respect to end effector 110, for instance, the likelihood that tissue contacting the end effector will be unintentionally thermally altered is reduced or eliminated. Accordingly, in at least one embodiment and referring again to FIG. 1, a surgical kit may include a surgical instrument, such as surgical instrument 100 described above, and an end effector cap, such as cap 280. The surgical instrument 100 may comprise end effector 110 that may also include at least one energy delivery surface, such as first and/or second energy delivery surfaces 175A and 175B (see FIG. 3). The end effector cap 110 may comprise a body 281 including a first end 283 and a second end 284. As seen in phantom lines in FIG. 1, in at least one embodiment, the body 281 may define a cavity 285 therein and the first end 283 may define an opening 282 to the cavity 285. Further, the second end 284 may be closed and/or sealed.

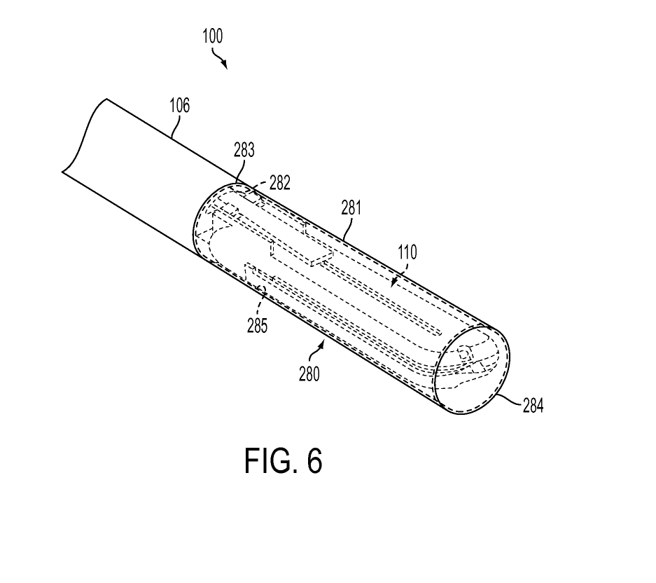

In at least one embodiment, the cavity 285 may sized and configured to receive at least a portion of the end effector 110. For example, referring to FIG. 1, the cap 280 is shown removed from the end effector 110. However, the jaws 120A and 120B may be closed as described above and shown in FIG. 4, and after closing the jaws, referring to FIG. 6, the cap 280 may be placed over the end effector 110 such that the end effector is positioned within the cavity 285. In at least one embodiment, the cap 280 may completely cover the end effector 110. In another embodiment, not illustrated, the cap 280 may partially cover the end effector 110. Additionally, the cap 280 may be held in place by an interference or press fit configuration. In such embodiments, the cavity 285 may be dimensioned such that the body 281 is configured to interference fit onto the end effector 110 and/or the elongate shaft 108. In such embodiments, the cap 280 may be held to the end effector 110 and/or the shaft 108 owing to friction between at least a portion of the end effector 110 and/or shaft 108 and the cap 280. However, the cap 280 may be removed from the end effector 110 when a user applies sufficient pulling force to the cap 280 to pull the cap 280 from the end effector 110 and/or elongate shaft 108.

In use, according to at least one embodiment, the surgical instrument 100 may be used as described above to deliver energy to tissue, thereby welding the tissue, for example. After such welding though, the temperatures of various components of the end effector 110, such as jaws 120A and 120B and/or energy delivery surfaces 175A and 175B, for example, may be high such that tissue and/or other items contacting such components may be thermally altered, undesirably. Thus, to help reduce or prevent such unintended thermal incidents, upon removing the instrument 100 from the patient, the cap 280 may be placed over the end effector 110, thereby covering the end effector 280, or at least the portions of the end effector 280 that may have a high temperature.

Additionally, in at least one embodiment, the cap 280 may aid in the transfer of heat from the jaws 120A and 120B. In such embodiments, the body 281 may comprise at least one metal, such as aluminum, for example. Thus, the cap 280 may absorb heat from the jaws 120A and/or 120B and dissipate the heat across the relatively larger surface area of body 281 as compared to the exterior surface area of the jaws 120A and/or 120B, for example.

In at least one embodiment, it may be desirable to insert the end effector 110 into a patient's body cavity with the cap 280 attached to the end effector 110. Accordingly, the body 281 may be sized and configured to fit through a trocar (not shown). A trocar, which is well known in the art, may comprise a tube defining a lumen therein. In use, the trocar's tube may be placed through a patient's body wall into a body cavity. Thereafter, the end effector 110, covered by cap 280, may be inserted through the trocar's lumen into the body cavity. Further, elongate shaft 108 of the surgical instrument 100 may also pass at least partially into the trocar's lumen. Accordingly, in at least one embodiment, the cap body 281 may be sized such that it has the same or smaller outer diameter as the shaft's outer diameter and thus may fit through a same or larger trocar through which the shaft 108 and/or the end effector 110 were originally sized to fit. Alternatively, the cap body 281 may have a larger outer diameter than the elongate shaft's outer diameter. In any event, after inserting the end effector 110, with cap 280 releasably attached thereto, into a patient's body cavity, the cap 280 may be removed by another surgical instrument, such as a grasper, inserted through another trocar, for example. Then, the surgical instrument 100 may grasp, clamp, cut, and/or weld or otherwise apply energy to tissue as described above. After performing one or more of such surgical tasks, the grasper, for example, may be used to place the cap 280 back over the end effector 110, thereby protecting the patient from receiving undesired energy from the end effector 110 while a user is removing the end effector 110 from the patient's body cavity, through the trocar.

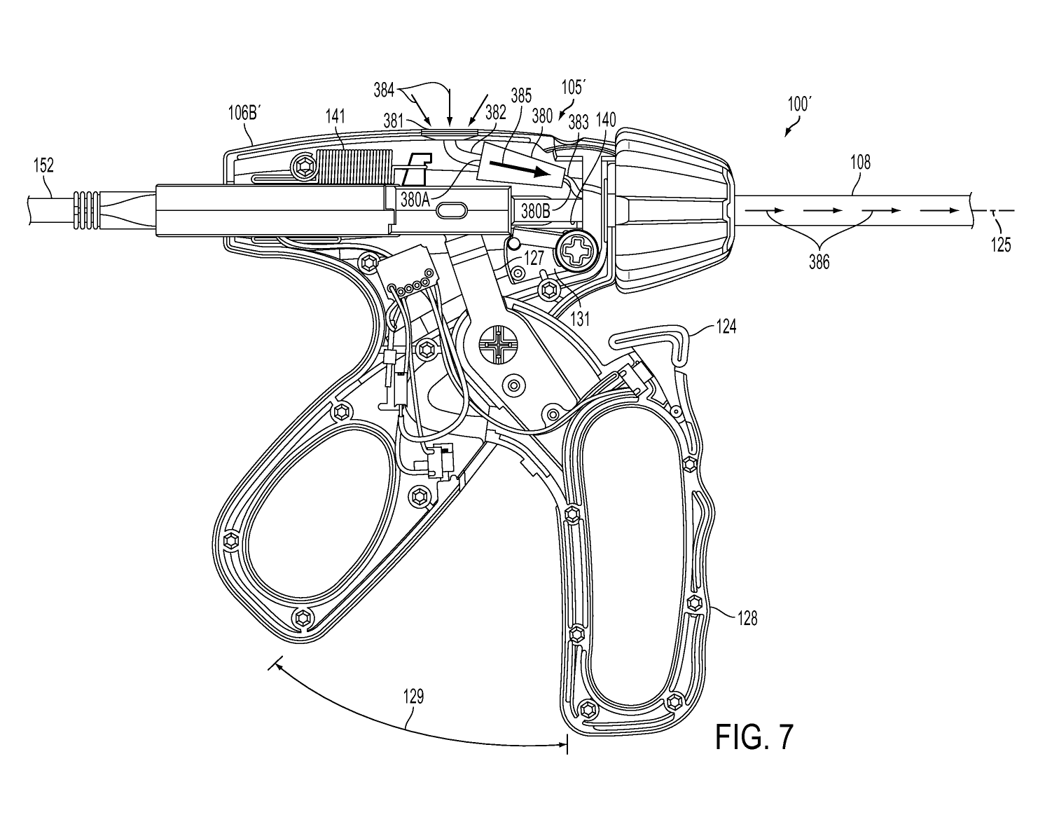

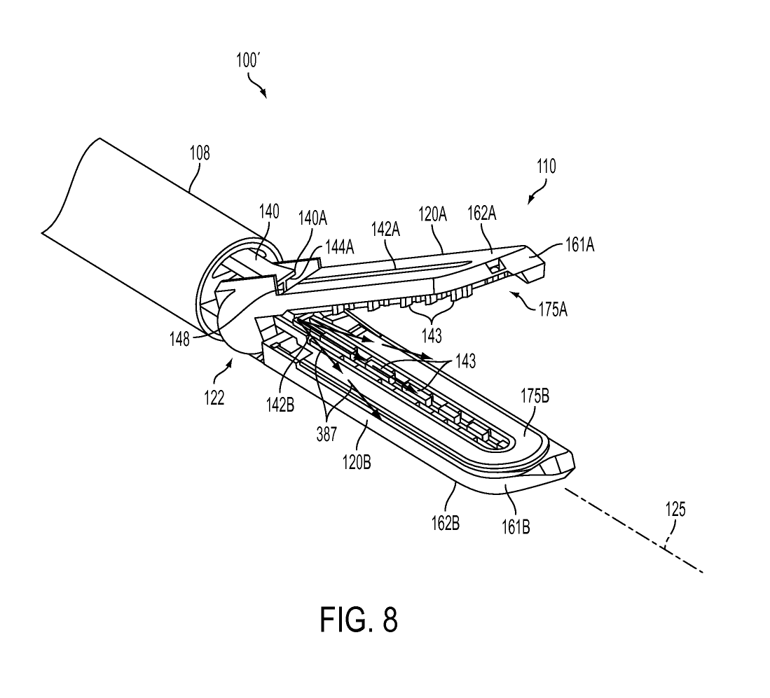

Other features may provide for the cooling of a surgical instrument's end effector. For example, in various embodiments, a surgical instrument may comprise a pump that is configured to cause a fluid to move over at least a portion of an end effector. More specifically, in at least one exemplary embodiment, referring now to FIGS. 7-8, a surgical instrument 100′ may be provided that comprises a pump 380 operably coupled to a handle 105′. Surgical instrument 100′ may be generally similar to surgical instrument 100 described above. For example, the handle 105′ may be operably coupled to an end effector 110 via an elongate shaft 108, as discussed above. Similarly, as discussed above, the handle 105′ may comprise a body including a first handle body (not illustrated) and second handle body 106B′. FIG. 7 shows the first handle body removed to show the components of surgical instrument 100′ associated with and/or within handle 105′. As illustrated, the pump 380 may be coupled to part of the handle body, such as second handle body 106B′. However, while pump 380 is shown located within handle 105′, the pump 380 may alternatively be positioned external to the handle 105′. In any event, the pump 380 may be configured to cause a fluid to move through the elongate shaft 108 and over at least a portion of the end effector 110. In at least one embodiment, the fluid may be a gas, such as air, for example.

In more detail, referring still to FIGS. 7-8, the handle 105′ may additionally comprise a fluid port 381 located on the body. The fluid port 381 may comprise a vent, for example, through which air from outside the instrument may pass. Additionally, in at least one embodiment, the fluid port 381 may comprise a filter, such as a HEPA air filter, to purify the air passing therethrough. The pump 380 may comprise an inlet 380A and an outlet 380B for the fluid to enter and exit the pump 380, respectively. In at least one embodiment, the inlet 380A may be coupled to the fluid port 381 via first tubing 382 and the outlet 380B may be coupled to the elongate shaft 380B by second tubing 383.

In use, the surgical instrument 100′, may function as follows. In at least one embodiment, referring to FIG. 7, when activation button 124 is pressed to supply energy to the end effector 110, as discussed above, the pump 380 may simultaneously or shortly thereafter activate. In such embodiments, the pump 380 may be connected to the activation button 124 by at least one electrical conductor (not shown), such as an electrical lead, insulated wire, and/or copper wire, for example. Accordingly, the button 124 may be configured to be moved between a first and a second position where the second position completes an electrical circuit such that current may flow from a power source outside the instrument, such as that associated with controller 150 and/or electrical source 145, for example, to the pump 380. Thus, in at least one embodiment, when the button 124 is depressed to the second position, electrical current may flow from the electrical source 145, for example, through the electrical conductors (not shown), to the pump 380. The pump 380 may thereby activate and begin to draw air, designated by arrows 384, into fluid port 381, through first tubing 382 and into pump 380 via inlet 380A. The pump 380 may continue to force the air, designated by arrow 385, out outlet 380B, into second tubing 383 and into elongate shaft 108. The air, designated by arrows 386, may then travel in a distal direction through the elongate shaft 108 and toward the end effector 110, see FIG. 8. Referring now to FIG. 8, the air, designated by arrows 387, may thereafter be forced over at least a portion of the end effector 110. As illustrated in FIG. 8, the air may enter the space between jaws 120A and 120B, thereby allowing for the energy delivery surfaces 175A and 175B to be subsequently cooled. Alternatively or additionally, the fluid may be forced over the outer surfaces of one or both of the jaws 120A and 120B (see FIG. 13, discussed below).

While the pump 380 may be configured to operate during a surgical procedure by being activated at or at about the same time as energy is delivered to surfaces 175A and/or 175B, the pump may be configured to be selectively activated independently of the energy delivery activation button's use. Referring to FIG. 7, in at least one embodiment, the pump may alternatively be coupled to a control button (not shown) on the exterior of the handle 105′. In such embodiments, the pump 380 may be activated before, during, and/or after a surgical procedure by pressing the control button, thereby allowing for selective cooling of the end effector 110 (see FIG. 8) before, during, and/or after a surgical operation.

As discussed above, a surgical instrument may comprise a pump that is configured to cause a fluid to move over at least a portion of an end effector as described above, e.g., by forcing or pushing a fluid, such as a gas, such as air, for example, in a distal direction over part of the end effector. Alternatively, in various embodiments, a surgical instrument may comprise a pump that is configured to force or draw a fluid in a proximal direction over part of the end effector. In other words, a pump may be configured to function like a vacuum and draw one or more fluids into the end effector and then into the pump.

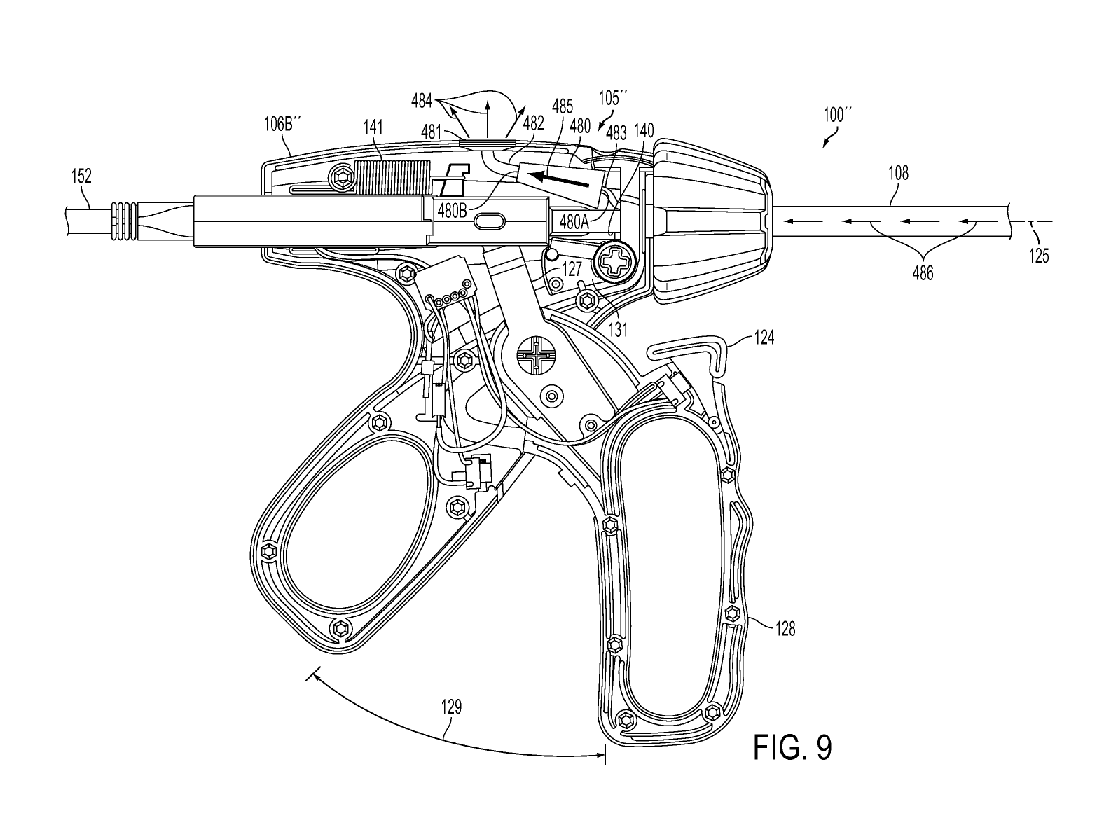

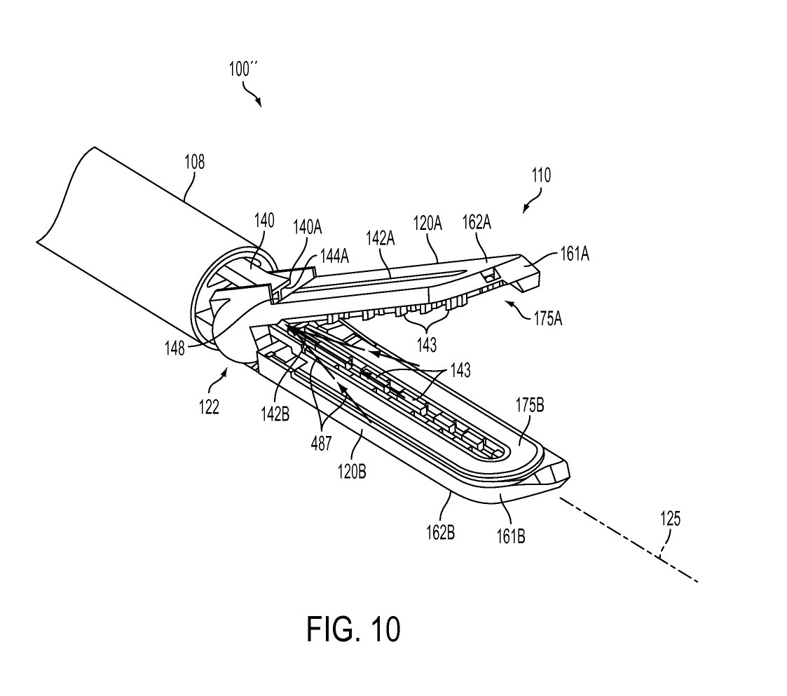

More specifically, in at least one exemplary embodiment, referring now to FIGS. 9-10, a surgical instrument 100″ may be provided that comprises a pump 480 operably coupled to a handle 105″. Surgical instrument 100″ may be generally similar to surgical instrument 100 described above. For example, the handle 105″ may be operably coupled to an end effector 110 via an elongate shaft 108, as discussed above. Similarly, as discussed above, the handle 105″ may comprise a body including a first handle body (not illustrated) and second handle body 106B″. FIG. 9 shows the first handle body removed to show the components of surgical instrument 100″ associated with and/or within handle 105″. As illustrated, the pump 480 may be coupled to part of the handle body, such as second handle body 106B″. However, while pump 480 is shown located within handle 105″, the pump 480 may alternatively be positioned external to the handle 105″. In any event, the pump 480 may be configured to cause a fluid to move over at least a portion of the end effector 110 and through the elongate shaft 108. In at least one embodiment, the fluid may be a gas, such as air, for example.

In more detail, referring still to FIGS. 9-10, the handle 105″ may additionally comprise a fluid port 481 located on the body. The fluid port 481 may comprise a vent, for example, through which air from the instrument may pass and/or be exhausted. Additionally, in at least one embodiment, the fluid port 481 may comprise a filter, such as a HEPA air filter, to purify the air passing therethrough. The pump 480 may comprise an inlet 480A and an outlet 480B for the fluid to enter and exit the pump 480, respectively. In at least one embodiment, the inlet 480A may be coupled to the elongate shaft 108 by second tubing 483, and the outlet 480B may be coupled to the fluid port 481 by first tubing 482.

In use, the surgical instrument 100″, may function as follows. In at least one embodiment, referring to FIG. 9, when activation button 124 is pressed to supply energy to the end effector 110, as discussed above, the pump 480 may simultaneously or shortly thereafter activate. In such embodiments, the pump 480 may be connected to the activation button 124 by at least one electrical conductor (not shown), such as an electrical lead, insulated wire, and/or copper wire, for example. Accordingly, the button 124 may be configured to be moved between a first and a second position where the second position completes an electrical circuit such that current may flow from a power source outside the instrument, such as that associated with controller 150 and/or electrical source 145, for example, to the pump 480. Thus, in at least one embodiment, when the button 124 is depressed to the second position, electrical current may flow from the electrical source 145, for example, through the electrical conductors (not shown), to the pump 380. Referring now to FIGS. 9 and 10, the pump 480 may thereby activate and begin to draw air, designated by arrows 487, into end effector 110 and/or elongate shaft 108. The air, designated by arrows 486, may then travel in a proximal direction through the elongate shaft 108 and toward the handle 105″. The pump 480 may continue to force the air through the second tubing 482 and into pump 480 via inlet 480A. The air, designated by arrow 485, may then be forced out outlet 480B, into first tubing 483 and toward fluid port 481. Thereafter, the air, designated by arrows 484, may be forced out fluid port 481 and into the environment outside the surgical instrument 100. Accordingly, referring to FIG. 10, the air may be forced over at least a portion of the end effector 110 by a vacuum-like effect created by the pump 480. As illustrated in FIG. 10, the air may pass through the space between jaws 120A and 120B, thereby allowing for the energy delivery surfaces 175A and 175B to be subsequently cooled. Also, although not illustrated, alternatively or additionally, the fluid may be drawn over the outer surfaces of one or both of the jaws 120A and 120B as discussed above with respect to surgical instrument 100′ and shown in FIG. 13 (albeit with the directional arrows in FIG. 13 pointing in a proximal, as opposed to a distal, direction). Additionally, in at least one embodiment, tissue, tissue fragments, and/or bodily fluids, instead of or in addition to air, may be vacuumed into end effector 110 and/or elongate shaft 108 via use of a pump, such as pump 480, that is configured to create suction at or near the end effector 110.

While the pump 480 may be configured to operate during a surgical procedure by being activated at or at about the same time as energy is delivered to surfaces 175A and/or 175B, the pump may be configured to be selectively activated independently of the energy delivery activation button's use. Referring to FIG. 9, in at least one embodiment, the pump may alternatively be coupled to a control button (not shown) on the exterior of the handle 105″. In such embodiments, the pump 480 may be activated before, during, and/or after a surgical procedure by pressing the control button, thereby allowing for selective cooling of the end effector 110, among other things, see FIG. 10, before, during, and/or after a surgical operation.

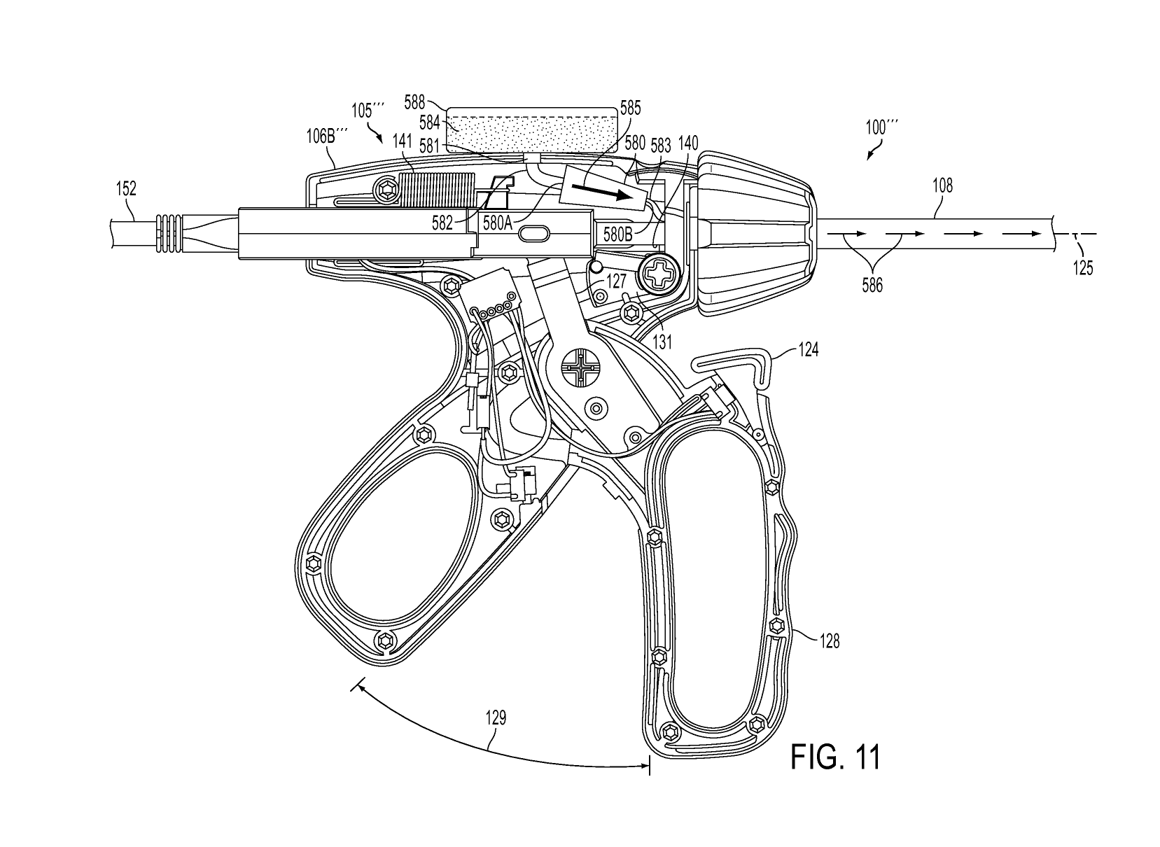

Various embodiments described above have utilized a pump to cause a fluid, such as a gas, e.g., air, for example to move over at least a portion of an end effector. Alternatively, the fluid may comprise a liquid, such as a saline solution, for example. More specifically, in at least one exemplary embodiment, referring now to FIGS. 11-13, a surgical instrument 100′″ may be provided that comprises a pump 580 operably coupled to a handle 105′″. Surgical instrument 100′″ may be generally similar to surgical instrument 100 described above. For example, the handle 105′″ may be operably coupled to an end effector 110 via an elongate shaft 108, as discussed above. Similarly, as discussed above, the handle 105′″ may comprise a body including a first handle body (not illustrated) and second handle body 106B′″. FIG. 11 shows the first handle body removed to show the components of surgical instrument 100′″ associated with and/or within handle 105′″. As illustrated, the pump 580 may be coupled to part of the handle body, such as second handle body 106B′″. However, while pump 580 is shown located within handle 105′″, the pump 580 may alternatively be positioned external to the handle 105′″. In any event, the pump 580 may be configured to cause a fluid to move through the elongate shaft 108 and over at least a portion of the end effector 110. As noted above, in at least one embodiment, the fluid may be a liquid, such as a saline solution, for example.

In more detail, referring to FIG. 11, the handle 105′″ may additionally comprise a fluid port 581 located on the body through which liquid may pass. Additionally, in at least one embodiment, the surgical instrument 100′″ may further comprise a fluid reservoir, such as fluid reservoir 588, for example, that is operably coupled to the fluid port 581. The reservoir may hold a liquid 584 therein. Again, among other things, the liquid 584 may comprise a saline solution and/or other biocompatible liquid, for example. In more detail, in at least one embodiment, the reservoir 588 may be coupled to the port 581 by way of a luer lock connection. For example, the fluid port 581 may comprise a female luer lock connector and the fluid reservoir 588 may comprise a male luer lock connector. The two luer lock connectors may be sized and configured to releasably mate and/or seal with each other via a twisting action therebetween. Accordingly, the fluid reservoir may be removed from the body 105′″ if so desired, such as to allow a replacement reservoir to be substituted for a spent reservoir in a situation were additional saline solution is needed during a surgical procedure, for example. Alternatively, the reservoir 588 may be fixedly connected and/or sealed to the port 581 to allow for a single use instrument 100′″ and/or fluid reservoir 588.

In any event, in at least one embodiment, the pump 580 may comprise an inlet 580A and an outlet 580B for the fluid to enter and exit the pump 580, respectively. In at least one embodiment, the inlet 580A may be coupled to the fluid port 581 via first tubing 582 and the outlet 580B may be coupled to the elongate shaft 580B by second tubing 583.

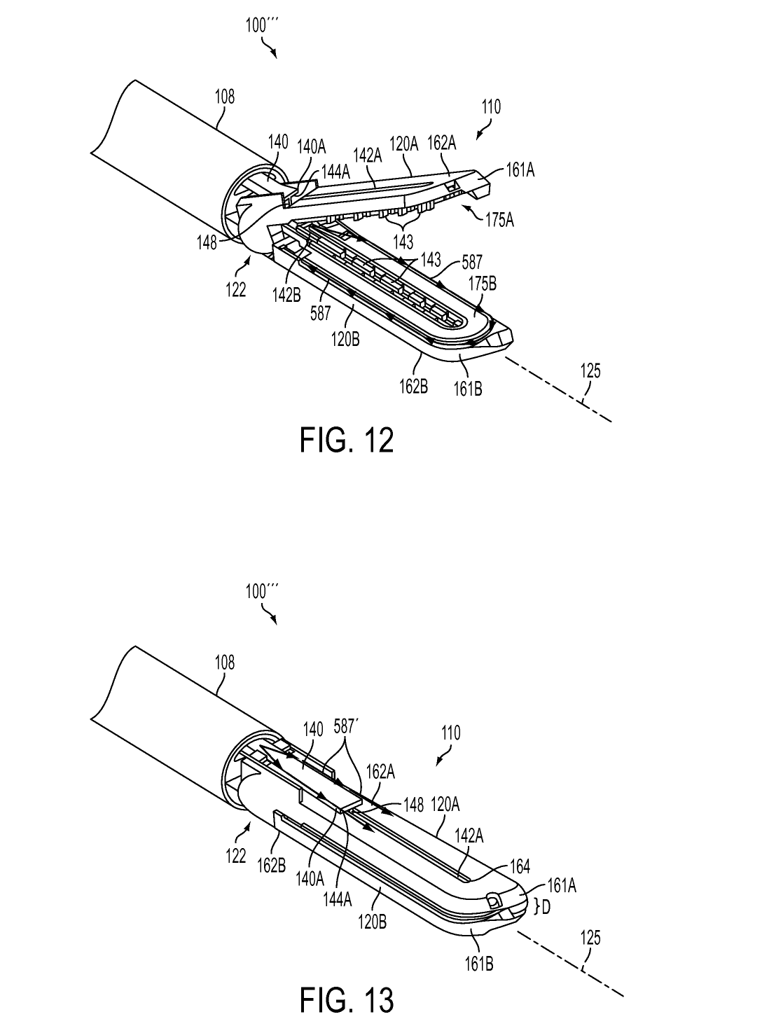

In use, the surgical instrument 100′″, may function as follows. In at least one embodiment, referring to FIG. 11, when activation button 124 is pressed to supply energy to the end effector 110, as discussed above, the pump 580 may simultaneously or shortly thereafter activate. In such embodiments, the pump 580 may be connected to the activation button 124 by at least one electrical conductor (not shown), such as an electrical lead, insulated wire, and/or copper wire, for example. Accordingly, the button 124 may be configured to be moved between a first and a second position where the second position completes an electrical circuit such that current may flow from a power source outside the instrument, such as that associated with controller 150 and/or electrical source 145, for example, to the pump 580. Thus, in at least one embodiment, when the button 124 is depressed to the second position, electrical current may flow from the electrical source 145, for example, through the electrical conductors (not shown), to the pump 580. The pump 580 may thereby activate and begin to draw liquid 584 into fluid port 581, through first tubing 582 and into pump 580 via inlet 580A. The pump 580 may continue to force the liquid, in a direction generally designated by arrow 585, out outlet 580B, into second tubing 583 and into elongate shaft 108. The liquid, moving in a direction generally designated by arrows 586, may then travel in a distal direction through the elongate shaft 108 and toward the end effector 110, see FIGS. 12 and 13. Referring now to FIGS. 12 and 13, the liquid, moving in a direction generally designated by arrows 587 and 587′, respectively, may thereafter be forced over at least a portion of the end effector 110. The liquid may move through at least a portion of one or both of jaws 120A and 120B, thereby allowing for the energy delivery surfaces 175A and 175B to be subsequently cooled. In at least one embodiment, referring to FIG. 12, one or both of jaws 120A and 120B may include a small channel along the perimeter of the energy delivery surfaces 175A and/or 175B such that a liquid path is defined around at least a portion of one or both of the surfaces perimeters. With respect to jaw 120B, the liquid may be directed and travel along the path, generally in the direction of arrows 587 as best seen in FIG. 12. Such a path may allow for better heat transfer between the energy delivery surfaces 175A and/or 175B and the liquid passing thereby while still permitting energy to flow between surfaces 175A and 175B such that tissue is effectively welded therebetween, as discussed above. Accordingly, tissue may be welded while preventing or resisting overheating of the jaws 120A and 120B. Alternatively or additionally, when the jaws 120A and 120B are in a closed position as shown in FIG. 13, the fluid may be forced over the outer surfaces of one or both of the jaws 120A and 120B, such as over outward-facing surfaces 162A and/or 162B, in a direction generally designated by arrows 587′, for example.

While the pump 580 may be configured to operate during a surgical procedure by being activated at or at about the same time as energy is delivered to surfaces 175A and/or 175B, the pump may be configured to be selectively activated independently of the energy delivery activation button's use. Referring to FIG. 11, the pump may alternatively be coupled to a control button (not shown) on the exterior of the handle 105′″. In such embodiments, the pump 580 may be activated before, during, and/or after a surgical procedure by pressing the control button, thereby allowing for selective cooling of the end effector 110, see FIGS. 12 and/or 13, before, during, and/or after a surgical operation. If the pump 580 is activated post-operatively, liquid may be expelled from the end effector 110 as the user removes the instrument from a patient's body.

Among other things, various cooling means have been described above for cooling at least a portion of an end effector of a surgical instrument. For example, such cooling means thus far described include a protective end effector cap 280 (see FIGS. 1 and 6) and/or one or more pumps, such as pumps 380, 480, and/or 580 (see FIGS. 7, 9, and 11, respectively), and/or the pumps' respective related components, which are configured to cause a fluid to move over at least a portion of an end effector. However, additional cooling means, used independently, or in addition to one or more of the above described cooling means, may also provide for enhanced cooling of at least a portion of an end effector. Accordingly, in various embodiments, referring again to FIGS. 1 and 3, a surgical instrument, such as surgical instrument 100 seen in FIG. 1, may comprise an end effector 110 comprising at least one energy delivery surface, such as one or both energy delivery surfaces 175A and 175B seen in FIG. 3, and a cooling means for cooling at least a portion of the end effector.

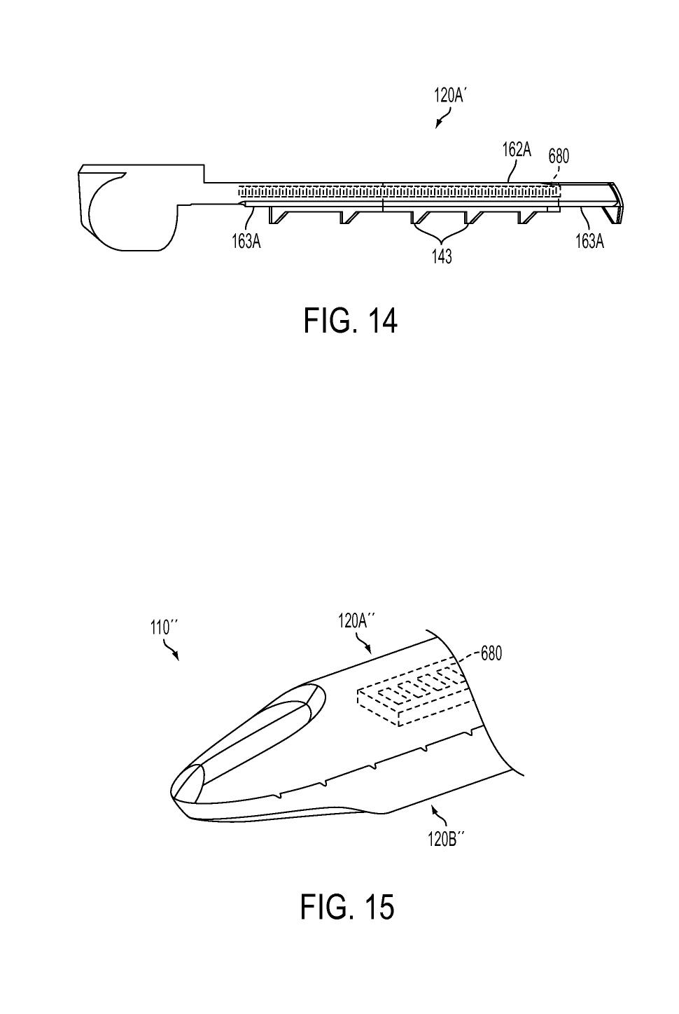

In at least one embodiment, referring now to FIGS. 1 and 14, the cooling means may comprise a heat sink 680 located within the end effector 110. The end effector 110 may comprise an upper first jaw 120A′ similar to jaw 120A described above. However, as illustrated in FIG. 14, the heat sink 680 may be located within the upper first jaw 120A′ and, although not shown, a second heat sink may be located within a similarly modified variant of lower second jaw 120B seen in FIG. 3, for example. FIG. 15 shows a partial perspective view of an end effector 110″ comprising jaws 120A″ and 120B″ and the relative location of heat sink 680 within jaw 120A″. In any event, the heat sink 680 may be positioned, sized, and configured to absorb and dissipate heat as the surgical instrument 100 (see FIG. 1) is used and/or as energy is delivered to one or both of surfaces 175A and 175B (see FIG. 3).

Additional cooling means are also possible. For example, in at least one embodiment, referring to FIG. 4, the gap or distance between the jaws 120A, 120B may be increased to aid in cooling the end effector 110. However, in such embodiments, the gap or dimension D between energy delivery surfaces 175A and 175B may provide for a satisfactory tissue weld and/or hemostasis effect, as discussed above. Thus, while dimension D may equal about 0.0005″ to about 0.005″, for example, and preferably between about 0.001″ to about 0.002″, for example, the distance between the jaws 120A and 120B may be greater than the dimension D. Accordingly, this larger distance between the jaws may provide greater exposure to the energy delivery surfaces 175A and 175B to increase heat dissipation therefrom. Additionally or alternatively, the energy delivery surfaces 175A and 175B may be increased in size to provide for additional cooling and temperature control by maximizing the amount of exposed energy delivery surface area.

While the cooling means may be passive as in the case of a protective cap (after it is positioned over an end effector), heat sink, and/or geometric configuration of the jaws and/or energy delivery surfaces described above, cooling means such as those including a pump, also described above, may be active in that the respective cooling means may be selectively activated, actuated, energized, or otherwise caused, by a user or other mechanism, to effect cooling of an end effector. Further, in various embodiments, a sensor, such as a thermocouple, for example, may be utilized to actively control the flow of energy to the end effector.

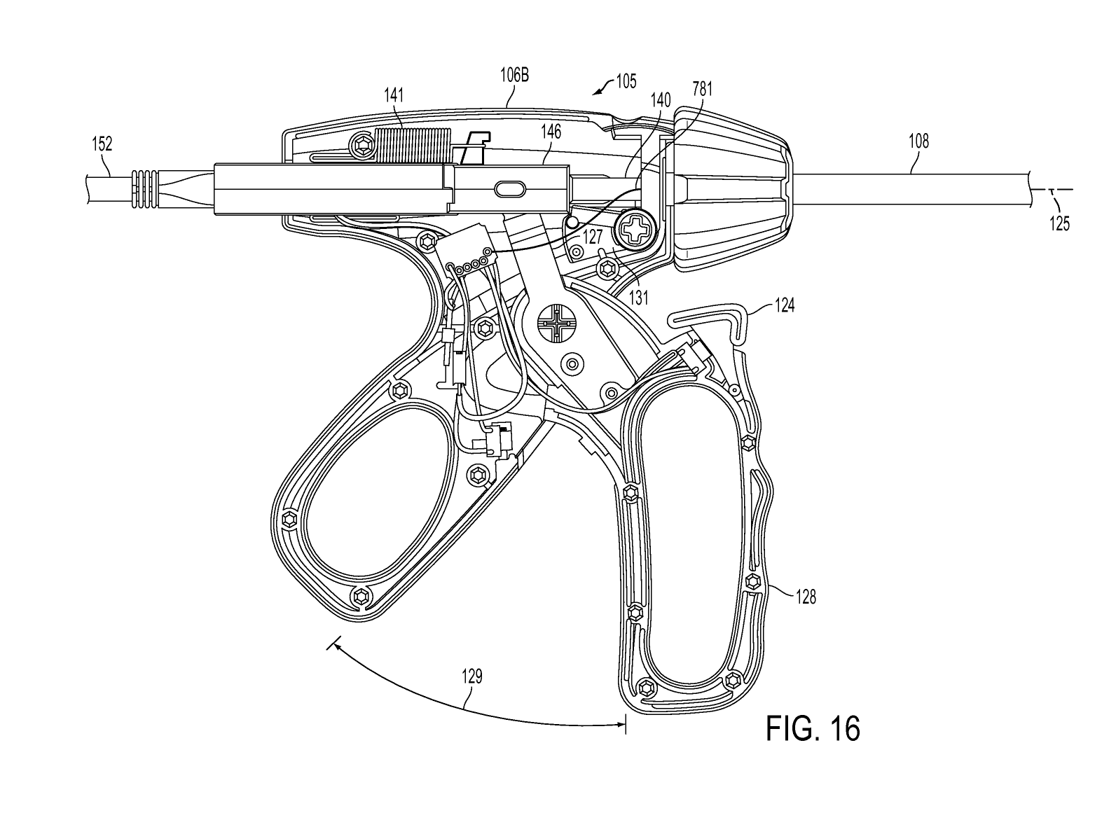

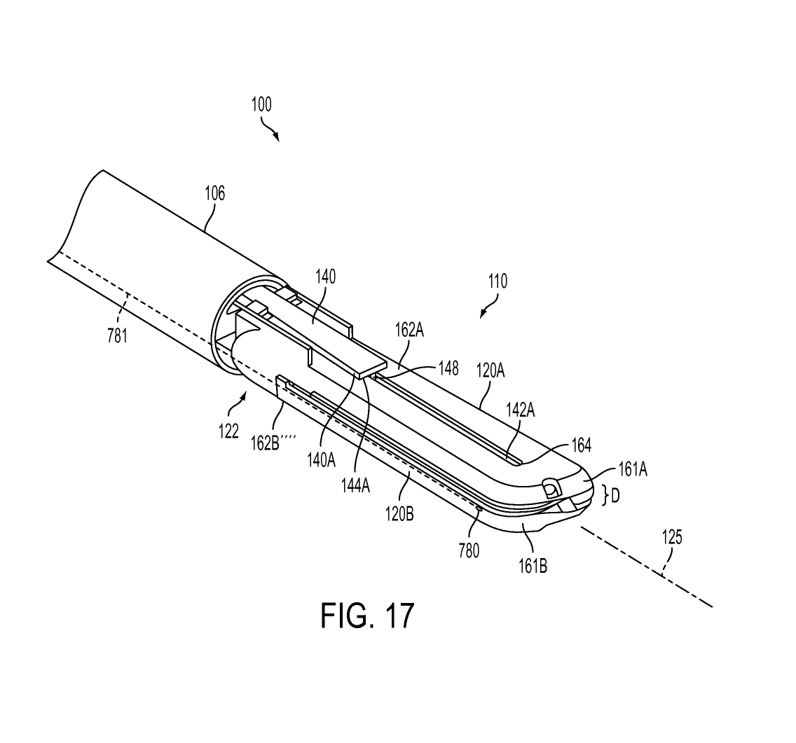

For example, in at least one embodiment and referring to FIGS. 16-17, the cooling means may comprise a thermocouple 780 located within the end effector 110 and an electrical conductor 781 extending from the thermocouple 780. The electrical conductor 781 may comprise an electrical lead, insulated wire and/or copper wire, for example, and may be configured to couple the thermocouple 780 to a controller and an energy source, such as controller 150 and electrical source 145 (see FIG. 1), for example. The conductor 781 may extend from the thermocouple 780, pass out the end effector 110, and extend through the elongate shaft 108 into handle 105 where it may be electrically coupled to cable 152. As noted above, the cable 152 may be electrically coupled to controller 150 and electrical source 145. In at least one embodiment, the controller 150 may modulate the energy produced by the energy source 145 in response to signal feedback provided by the thermocouple 780. Accordingly, the energy delivered to end effector 110 may be regulated by the temperature sensed by the thermocouple 780 in end effector 110. In more detail, when activation button 124 is depressed, the controller 150 may be programmed and configured to deactivate or reduce the output of the electrical source 145 when the temperature around the thermocouple 780 is above a certain predetermined threshold and to reactivate or increase the output of the electrical source 145 when the temperature around the thermocouple 780 is below a certain predetermined threshold, thereby helping prevent or resist overheating of the end effector 110 by automatically controlling the amount of energy flowing to and/or through the end effector 110 in accordance with the temperature of the end effector 110, at or around thermocouple 780.

The embodiments of the devices described herein may be introduced inside a patient using minimally invasive or open surgical techniques. In some instances it may be advantageous to introduce the devices inside the patient using a combination of minimally invasive and open surgical techniques. Minimally invasive techniques may provide more accurate and effective access to the treatment region for diagnostic and treatment procedures. To reach internal treatment regions within the patient, the devices described herein may be inserted laparoscopically, such as in a multiple site laparoscopy, a single site laparoscopy, or a single incision laparoscopic surgery, for example. Further, the devices described here may be used in a a single port access procedure, for example. Additionally or alternatively, the devices described herein may be inserted through natural openings of the body such as the mouth, anus, and/or vagina, for example. Minimally invasive procedures performed by the introduction of various medical devices into the patient through a natural opening of the patient are known in the art as NOTES™ procedures. Some portions of the devices may be introduced to the tissue treatment region percutaneously or through small-keyhole-incisions.

Endoscopic minimally invasive surgical and diagnostic medical procedures are used to evaluate and treat internal organs by inserting a small tube into the body. The endoscope may have a rigid or a flexible tube. A flexible endoscope may be introduced either through a natural body opening (e.g., mouth, anus, and/or vagina) or via a trocar through a relatively small-keyhole-incision incisions (usually 0.5-1.5 cm). The endoscope can be used to observe surface conditions of internal organs, including abnormal or diseased tissue such as lesions and other surface conditions and capture images for visual inspection and photography. The endoscope may be adapted and configured with working channels for introducing medical instruments to the treatment region for taking biopsies, retrieving foreign objects, and/or performing surgical procedures.

The devices disclosed herein may be designed to be disposed of after a single use, or they may be designed to be used multiple times. In either case, however, the device may be reconditioned for reuse after at least one use. Reconditioning may include a combination of the steps of disassembly of the device, followed by cleaning or replacement of particular pieces, and subsequent reassembly. In particular, the device may be disassembled, and any number of particular pieces or parts of the device may be selectively replaced or removed in any combination. Upon cleaning and/or replacement of particular parts, the device may be reassembled for subsequent use either at a reconditioning facility, or by a surgical team immediately prior to a surgical procedure. Those of ordinary skill in the art will appreciate that the reconditioning of a device may utilize a variety of different techniques for disassembly, cleaning/replacement, and reassembly. Use of such techniques, and the resulting reconditioned device, are all within the scope of this application.

Preferably, the various embodiments of the devices described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility. Other sterilization techniques can be done by any number of ways known to those skilled in the art including beta or gamma radiation, ethylene oxide, and/or steam.

Although the various embodiments of the devices have been described herein in connection with certain disclosed embodiments, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

1. A surgical kit, comprising:
a surgical instrument comprising an end effector; and
a cap comprising a body including a first end and a second end, wherein the body defines a cavity and the first end defines an opening to the cavity, wherein the cavity is sized and configured to receive at least a portion of the end effector, and wherein the body is sized and configured to fit through a trocar.

2. The surgical kit of claim 1, wherein the body comprises at least one metal.

3. The surgical kit of claim 2, wherein the at least one metal comprises aluminum.

4. The surgical kit of claim 1, wherein the end effector comprises at least one energy delivery surface.

5. The surgical kit of claim 1, wherein the cavity is dimensioned such that the body is configured to interference fit onto the end effector.

6. A surgical instrument, comprising:
a handle;
an end effector; and
an elongate shaft operably coupling the handle to the end effector; and
a pump operably coupled to the handle, wherein the pump is configured to cause a fluid to move through the elongate shaft and over at least a portion of the end effector.

7. The surgical instrument of claim 6, wherein the pump is located within the handle.

8. The surgical instrument of claim 6, wherein the handle comprises a body and a fluid port located on the body, wherein the pump comprises an inlet and an outlet, wherein the inlet is operably coupled to the fluid port, and wherein the outlet is operably coupled to the elongate shaft.

9. The surgical instrument of claim 6, wherein the handle comprises a body and a fluid port located on the body, wherein the pump comprises an inlet and an outlet, wherein the inlet is operably coupled to the elongate shaft, and wherein the outlet is operably coupled to the fluid port.

10. The surgical instrument of claim 6, wherein the fluid comprises a gas.

11. The surgical instrument of claim 10, wherein the gas comprises air.

12. The surgical instrument of claim 6, wherein the fluid comprises a liquid.

13. The surgical instrument of claim 12, wherein the liquid comprises a saline solution.

14. The surgical instrument of claim 12, wherein the end effector comprises at least one energy delivery surface including a perimeter and wherein the end effector is configured to direct the liquid around at least a portion of the energy delivery surface's perimeter.

15. The surgical instrument of claim 6, further comprising a fluid reservoir, wherein the handle comprises a body and a fluid port located on the body, wherein the fluid reservoir is operably coupled to the fluid port.

16. The surgical instrument of claim 15, wherein the pump comprises an inlet and an outlet, wherein the inlet is operably coupled to the fluid port, and wherein the outlet is operably coupled to the elongate shaft.

17. A surgical instrument, comprising:
an end effector comprising at least one energy delivery surface; and
cooling means for cooling at least a portion of the end effector.

18. The surgical instrument of claim 17, wherein the cooling means comprises a heat sink located within the end effector.

19. The surgical instrument of claim 17, wherein the cooling means comprises:
a thermocouple located within the end effector; and
an electrical conductor extending from the thermocouple, wherein the electrical conductor is configured to be coupled to a controller and an energy source, wherein the controller modulates energy produced by the energy source in response to a signal produced by the thermocouple.

20. The surgical instrument of claim 17, wherein the cooling means comprises active cooling means for actively cooling at least a portion of the end effector.

21. The surgical instrument of claim 17, wherein the cooling means comprises passive cooling means for passively cooling at least a portion of the end effector.

22. The surgical instrument of claim 6, further comprising a cutting member movably disposed at least partially within the elongate shaft, and wherein the pump is configured to cause the fluid to move alongside the cutting member.

23. The surgical instrument of claim 22, wherein the pump is located within the handle.

24. The surgical instrument of claim 22, wherein the handle comprises a body and a fluid port located on the body, wherein the pump comprises an inlet and an outlet, wherein the inlet is operably coupled to the fluid port, and wherein the outlet is operably coupled to the elongate shaft.

25. The surgical instrument of claim 22, wherein the handle comprises a body and a fluid port located on the body, wherein the pump comprises an inlet and an outlet, wherein the inlet is operably coupled to the elongate shaft, and wherein the outlet is operably coupled to the fluid port.

26. The surgical instrument of claim 22, wherein the fluid comprises a gas.

27. The surgical instrument of claim 26, wherein the gas comprises air.

28. The surgical instrument of claim 22, wherein the fluid comprises a liquid.

29. The surgical instrument of claim 28, wherein the liquid comprises a saline solution.

30. The surgical instrument of claim 28, wherein the end effector comprises at least one energy delivery surface including a perimeter and wherein the end effector is configured to direct the liquid around at least a portion of the energy delivery surface's perimeter.

31. The surgical instrument of claim 22, further comprising a fluid reservoir, wherein the handle comprises a body and a fluid port located on the body, wherein the fluid reservoir is operably coupled to the fluid port.

32. The surgical instrument of claim 31, wherein the pump comprises an inlet and an outlet, wherein the inlet is operably coupled to the fluid port, and wherein the outlet is operably coupled to the elongate shaft.
